(19) United States
(12) Patent Application Publication
(10) Pub. No.: US 2026/0033504 A1
(43) Pub. Date: Feb. 5, 2026

(54) PORTABLE UV STERILISING DEVICE WITH MACHINE LEARNING MODEL

(71) Applicant: SHAHEEN INNOVATIONS HOLDING LIMITED, Abu Dhabi (AE)

(72) Inventor: Mohammed Alshaiba Saleh Ghannam Almazrouei, Abu Dhabi (AE)

(21) Appl. No.: 19/279,765

(22) Filed: Jul. 24, 2025

Related U.S. Application Data
(60) Provisional application No. 63/679,538, filed on Aug. 5, 2024.
(60) Provisional application No. 63/679,544, filed on Aug. 5, 2024.
(60) Provisional application No. 63/701,244, filed on Sep. 30, 2024.
(60) Provisional application No. 63/701,257, filed on Sep. 30, 2024.

(51) Int. Cl. A23B 2/53; A61L 2/10; A61L 2/24

(52) U.S. Cl. CPC A23B 2/53; A61L 2/10; A61L 2/24; A61L 2202/11; A61L 2202/14; A61L 2202/16

(57) ABSTRACT

A portable UV sterilising device comprises a proximal portion configured to be held by a user, and a distal portion adjacent the proximal portion, a plurality of UV light sources positioned at the distal portion and spaced around the distal portion, the plurality of UV light sources configured to project UV light outside the distal portion, wherein when the plurality of UV light sources is activated the plurality of UV light sources is configured to project the UV light radially around substantially all of the distal portion, and a control unit configured to control at least one of exposure duration and UV light intensity for the plurality of UV light sources, wherein the control unit is configured to use at least one machine learning (ML) model to control at least one of the exposure duration and the UV light intensity.

### CROSS-REFERENCES TO RELATED APPLICATIONS

The present application claims the benefit of priority to: U.S. Provisional Patent Application No. 63/679,538, filed on 5 Aug. 2024; U.S. Provisional Patent Application No. 63/679,544, filed on 5 Aug. 2024; U.S. Provisional Patent Application No. 63/701,244, filed on 30 Sep. 2024; and U.S. Provisional Patent Application No. 63/701,257, filed on 30 Sep. 2024, the contents of which are incorporated herein by reference in their entirety.

### FIELD

The present invention relates to a portable UV sterilising device, and more particularly to a portable UV sterilising device for the germicidal treatment of both consumable and non-consumable products using a machine learning model.

### BACKGROUND

Pasteurisation is a process which is applied to food products, such as dairy products, to destroy harmful pathogens and prolong their shelf life. Conventional pasteurisation methods involve heating the food product to a particular temperature for a prescribed time. These methods are known as thermal pasteurisation.

In conventional thermal pasteurisation, the food product is heated to a temperature between 60-110° C. (140-230° F.). However, the food product may alternatively be heated to a higher temperature for a shorter period of time to provide an even longer shelf life.

There is evidence to suggest that there are disadvantages to conventional thermal pasteurisation of products. There may be detrimental effects on nutritional value, such as a reduction in vitamin concentrations. Additionally, there may be changes in smell and taste attributes.

In recent years, several non-thermal pasteurisation technologies are emerging, but these technologies are largely experimental in nature and heat is the pasteurisation method of choice for many food products commercially.

The use of UV light is a promising method for reducing microbial load in food and beverages without the heat-associated drawbacks. However, existing solutions are often industrial-scale and complex, and ultimately not suitable for domestic or small-scale commercial use.

There is a need for a device that alleviates at least some of the problems outlined herein.

### BRIEF DESCRIPTION OF THE INVENTION

The present invention provides a portable UV sterilising device as claimed in claim 1, a system as claimed in claim 19, and a method as claimed in claim 20. The present invention also provides preferred features as claimed in the dependent claims.

### REPRESENTATIVE FEATURES

Representative features are set out in the following clauses, which stand alone or may be combined, in any combination, with one or more features disclosed in the text and/or drawings of the specification.
1. A portable UV sterilising device, the device comprising: a proximal portion configured to be held by a hand of a user; a distal portion coupled to the proximal portion; a plurality of UV light sources positioned at the distal portion and spaced around the distal portion, the plurality of UV light sources configured to project UV light outside the distal portion; a control unit positioned at least partly within the proximal portion, the control unit being electrically coupled to the plurality of UV light sources, and the control unit being configured to control the UV light sources to regulate an intensity and an exposure duration of the UV light projected by the UV light sources; and a battery, positioned at least partly within the proximal portion and electrically coupled to the control unit; wherein when the UV sterilising device is activated, the control unit controls the UV light sources to project UV light radially around at least a part of the distal portion.
2. The portable UV sterilising device of clause 1, wherein the proximal portion and the distal portion are at least partly of a UV light resistant material.
3. The portable UV sterilising device of clause 1 or 2, wherein the distal portion includes a rod and a protective casing which at least partly surrounds the rod, the UV light sources being positioned between the rod and the protective casing.
4. The portable UV sterilising device of any one of the preceding clauses, wherein at least part of the protective casing is translucent.
5. The portable UV sterilising device of any one of the preceding clauses, further comprising a removable cover configured to engage with the proximal portion and cover at least part of the distal portion, the cover including a slot configured to allow the passage of UV light in a predetermined direction when the cover is engaged with the proximal portion.
6. The portable UV sterilising device of any one of the preceding clauses, further comprising an integrated cooling system which is configured to dissipate heat generated by the UV light sources.
7. The portable UV sterilising device of any one of the preceding clauses, wherein the control unit includes a processor and a memory, the memory storing executable instructions which, when executed by the processor, cause the control unit to regulate the light intensity and exposure duration of the UV light sources using pulse width modulation.
8. The portable UV sterilising device of clause 7, wherein the control unit is configured to control the light intensity and exposure of the UV light sources based on a plurality of preset modes stored in the memory.
9. The portable UV sterilising device of clause 7 or 8, wherein the executable instructions include an executable instruction which, when executed by the processor, causes the control unit to alert the user to the sterilising device being activated.
10. The portable UV sterilising device of any one of clauses 7 to 9, wherein the executable instructions include an executable instruction which, when executed by the processor, causes the control unit to alert the user that the sterilising device has been activated for a predetermined length of time.
11. The portable UV sterilising device of any one of clauses 7 to 10, wherein the executable instructions include an executable instruction which, when executed by the processor, causes the control unit to alert the user that the sterilising device has been deactivated.
12. The portable UV sterilising device of any one of the preceding clauses, wherein the proximal portion includes a power button for activating the UV light sources.
13. The portable UV sterilising device of clause 11, wherein the executable instructions include an instruction which, when executed by the processor, causes the control unit to automatically deactivate the UV light sources if the power button is released.
14. The portable UV sterilising device of clause 12 or 13, wherein the executable instructions include an instruction which, when executed by the processor, causes the control unit to automatically deactivate the UV light sources after a predetermined time has elapsed since the UV light sources were activated.
15. The portable UV sterilising device of any one of clauses 12 to 14, wherein the proximal portion includes a sensor for detecting the microbial load in a medium to be sterilised, and wherein the executable instructions include an instruction which, when executed by the processor, causes the control unit to automatically deactivate the UV light sources once the microbial load is below a predetermined threshold.
16. The portable UV sterilising device of any one of clauses 12 to 15, wherein the proximal portion includes a depth sensor configured to detect the depth of the distal portion in a medium to be sterilised, and wherein the executable instructions include an instruction which, when executed by the processor, causes the control unit to automatically deactivate the UV light sources if the depth of the distal portion in the medium is decreased.
17. The portable UV sterilising device of any one of the preceding clauses, wherein the wavelength of the UV light is 100 nm to 280 nm.
18. The portable UV sterilising device of any one of the preceding clauses, wherein the wavelength of the UV light is approximately 254 nm.
19. A method for the germicidal treatment of a liquid in a vessel, the method comprising: providing a portable UV sterilising device incorporating: a proximal portion configured to be held by a hand of a user; a distal portion coupled to the proximal portion; a plurality of UV light sources positioned at the distal portion and spaced around the distal portion, the plurality of UV light sources configured to project UV light outside the distal portion; a control unit positioned at least partly within the proximal portion, the control unit being electrically coupled to the plurality of UV light sources, and the control unit being configured to control the UV light sources to regulate an intensity and an exposure duration of the UV light projected by the UV light sources; and a battery, positioned at least partly within the proximal portion and electrically coupled to the control unit, wherein the method further comprises: placing at least a part of the distal portion of the sterilising device in the vessel and submerging at least one of the UV light sources of the plurality of UV light sources in the liquid; controlling, using the control unit, the UV light sources to project UV light radially around at least part of the distal portion to treat the liquid in the; regulating, using the control unit, the intensity and the exposure duration of the UV light projected by the UV light sources; deactivating the UV light sources; and removing the distal portion from the vessel and out of the liquid.
20. The method of clause 19, further comprising stirring the liquid using at least part of the distal portion when the UV light sources are projecting the UV light radially around at least a part of the distal portion to treat the liquid in the vessel.
21. A method for the germicidal treatment of a non-liquid medium, the method comprising: providing a portable UV sterilising device according to clause 5; and passing the distal portion of the sterilising device over a surface of the medium.
22. A portable UV sterilising device comprising: a proximal portion configured to be held by a user, and a distal portion adjacent the proximal portion; a plurality of UV light sources positioned at the distal portion and spaced around the distal portion, the plurality of UV light sources configured to project UV light outside the distal portion; and a control unit electrically coupled to the plurality of UV light sources, the control unit configured to regulate the UV light intensity and exposure duration, wherein when the UV sterilising device is activated, UV light is projected radially, by the UV light source, around substantially all the distal portion.
23. A portable UV sterilising device comprising: a proximal portion configured to be held by a user, and a distal portion adjacent the proximal portion; a plurality of UV light sources positioned at the distal portion and spaced around the distal portion, the plurality of UV light sources configured to project UV light outside the distal portion, wherein when the plurality of UV light sources is activated the plurality of UV light sources is configured to project the UV light radially around substantially all of the distal portion; and a control unit configured to control at least one of exposure duration and UV light intensity for the plurality of UV light sources, wherein the control unit is configured to use at least one machine learning (ML) model to control at least one of the exposure duration and the UV light intensity.
24. The portable UV sterilising device of clause 23, wherein the control unit is configured to use the at least one ML model to control at least one of the exposure duration and the UV light intensity in dependence on input data indicative of at least one of: a type of subject to be treated, a quantity of the subject, and an initial microbial load associated with the subject.
25. The portable UV sterilising device of clause 24, wherein the subject is one of: food; a beverage; and a physical object comprising one or more surfaces.
26. The portable UV sterilising device of clause 25, wherein the physical object is one of: a laptop device, a mobile phone device, a keyboard device, and a work surface.
27. The portable UV sterilising device of any one of clauses 24 to 26, wherein the portable UV sterilising device comprises one or more sensors operable to detect at least some of the input data and/or an input interface operable to receive user input for specifying at least some of the input data.
28. The portable UV sterilising device of clause 27, wherein the one or more sensors comprise one or more of: an audio sensor operable to detect speech input; and an image sensor operable to capture one or more images, in which at least one of the speech input and the one or more images is indicative of at least one of a type and a quantity for the subject.
29. The portable UV sterilising device of any one of clauses 24 to 28, wherein the portable UV sterilising device comprises wireless communication circuitry, wherein the wireless communication circuitry is operable to receive: at least some of the input data from a mobile user device via a wireless communications network; or one or more control signals for controlling at least one of the exposure duration and the UV light intensity, the one or more control signals being based on ML model outputs in response to the input data.
30. The portable UV sterilising device of any one of clauses 24 to 29, wherein the control unit is configured to use the at least one ML model to control the exposure duration and the UV light intensity differently for at least one of: i) different food types and/or different beverage types; and/or ii) different quantities of a same food type and/or different quantities of a same beverage type; and/or iii) different microbial loads.
31. The portable UV sterilising device of any one of clauses 24 to 30, wherein the at least one ML model is operable to predict, in dependence on at least the type and the quantity of the subject, a required battery charge for treatment of the subject.
32. The portable UV sterilising device of any one of clauses 23 to 31, wherein the control unit is configured to use the at least one ML model to control at least one of the exposure duration and the UV light intensity in dependence on input data indicative of a remaining battery charge for one or more batteries for powering the portable UV sterilising device.
33. The portable UV sterilising device of clause 32, wherein in response to the remaining battery charge being at or below a preset level, the control unit is configured to use the at least one ML model to control the exposure duration and the UV light intensity to optimise power consumption for treatment of a subject.
34. The portable UV sterilising device of clause 32, wherein the control unit is configured to use the at least one ML model to control the exposure duration and the UV light intensity to balance power consumption and total treatment time for treatment of a subject.
35. The portable UV sterilising device of clause 32 when dependent on clause 31, wherein the control unit is configured to use the at least one ML model to predict whether treatment of the subject is possible for the remaining battery charge, and wherein the control unit is operable to activate the plurality of UV light sources in dependence on whether treatment of the subject is possible for the remaining battery charge.
36. The portable UV sterilising device of clause 32 when dependent on clause 31, wherein in response to the predicted required battery charge being greater than the remaining battery charge and within a given range of the remaining battery charge, the control unit is operable to reduce the UV light intensity to allow lower UV light intensity treatment for the subject over an increased exposure duration.
37. The portable UV sterilising device of any one of clauses 23 to 36, wherein the portable UV sterilising device has a plurality of modes of operation, at least some of the plurality of modes of operation having at least a different UV light intensity, and wherein the control unit is configured to use the at least one ML model to select a mode from the plurality of modes to thereby control the UV light intensity.
38. The portable UV sterilising device of any one of clauses 23 to 37, wherein the portable UV sterilising device has a plurality of modes of operation, at least some of the plurality of modes of operation having at least a different exposure duration, and wherein the control unit is configured to use the at least one ML model to select a mode from the plurality of modes to thereby control the exposure duration.
39. The portable UV sterilising device according to any one of clauses 23 to 38, wherein the control circuitry is configured to use the at least one ML model and/or one or more other ML models to predict at least one of: change in performance for the portable UV sterilising device; and degradation of one or more components for the portable UV sterilising device.
40. The portable UV sterilising device according to clause 39, wherein the control circuitry is configured to use the at least one ML model and/or the one or more other ML models to predict maintenance for the portable UV sterilising device in advance of component degradation below a set level.
41. A system comprising: the portable UV sterilising device according to any one of clauses 23 to 40; and a mobile user device, wherein the mobile user device is operable to communicate with the portable UV sterilising device via a wireless communication, and wherein at least one of the portable UV sterilising device, the mobile user device, and a server accessible by the mobile user device is operable to run the at least one ML model, and wherein the mobile user device comprises one or more sensors and/or an input interface operable to acquire data for input to the ML model.
42. A method for a portable UV sterilising device, the portable UV sterilising device comprising: a proximal portion configured to be held by a user, and a distal portion adjacent the proximal portion; a plurality of UV light sources positioned at the distal portion and spaced around the distal portion, the plurality of UV light sources configured to project UV light outside the distal portion, wherein when the plurality of UV light sources is activated the plurality of UV light sources is configured to project the UV light radially around substantially all of the distal portion, and a control unit configured to control at least one of exposure duration and UV light intensity for the plurality of UV light sources, wherein the method comprises: controlling at least one of exposure duration and UV light intensity for the plurality of UV light sources using at least one machine learning (ML) model.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a diagrammatic perspective view of a UV sterilising device of an example of this disclosure;

FIG. 2 is a diagrammatic front view of a UV sterilising device of an example of this disclosure;

FIG. 3 is a diagrammatic perspective view of a UV sterilising device of an example of this disclosure;

FIG. 4 is a diagrammatic view of a use of a UV sterilising device of an example of this disclosure;

FIG. 5 is a diagrammatic view of a circuit for a UV sterilising device of an example of this disclosure;

FIG. 6 is a diagrammatic view of a circuit for a UV sterilising device of an example of this disclosure;

FIG. 7 is a diagrammatic view of a circuit for a UV sterilising device of an example of this disclosure;

FIG. 8 is a schematic diagram of a system comprising a UV sterilising device, a mobile device, and one or more servers; and

FIG. 9 is a schematic diagram of a UV sterilising device and a mobile device comprising sensors.

### DETAILED DESCRIPTION OF THE DISCLOSURE

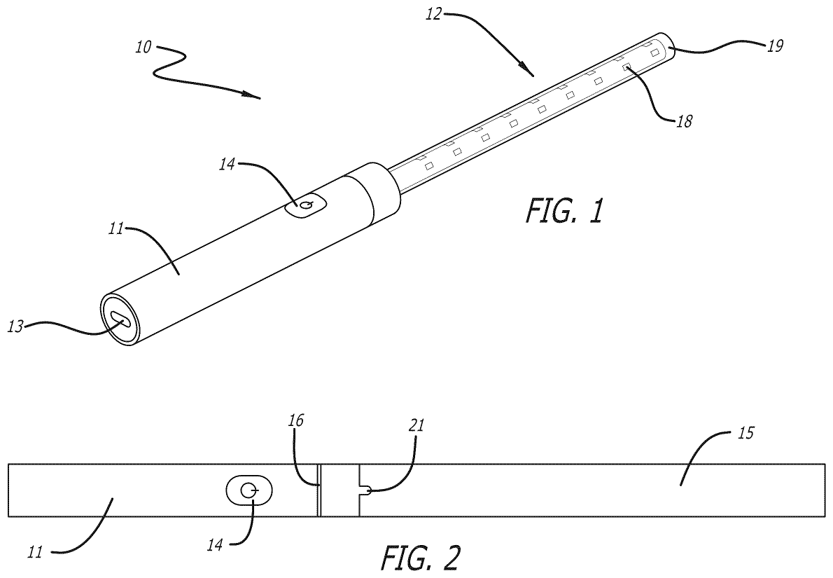

Referring to FIGS. 1 and 2, there is provided a UV sterilising device 10 according to some examples of the disclosure. Throughout the following description, the word “sterilising” is intended to encompass all forms of reducing the microbial load of a product, and is intended to specifically encompass “sanitising” a solid, as well as “pasteurising” a liquid.

The sterilising device 10 may be sized to be handheld and portable, and has two distinct portions: a first, proximal portion 11; and a second, distal portion 12 coupled to the proximal portion 11. At least some, and preferably all, of the exterior components are of a UV resistant material.

The proximal portion 11 of the sterilising device 10 is preferably of circular or oval cross-section, but may be of any shape. For example, the proximal portion 11 may have an ergonomically enhanced shape to aid gripping by a hand of a user. The proximal portion 11 may be of any material. Examples of suitable materials may be stainless steel or another metal, or a plastic material. The proximal portion 11 may be of, at least partially, a rubber, silicone, or other high friction material. The proximal portion 11, which may be the handle of the device, is thereby provided with a high grip surface so as to prevent slipping of the device in the hand of the user.

Further, the proximal portion 11 may be of a diameter and length so as to be comfortable to hold in a hand of a significant percentage of the population. The diameter and length may also be chosen such that components of the sterilising device 10 described below can be enclosed within the proximal portion 11.

The proximal portion 11 may function as a convenient housing for electronic components of the sterilising device 10, such as the control unit and related circuitry, a battery, one or more haptic motors, and one or more sensors. The proximal portion 11 may therefore be a thin, extruded shell of material. Ballast may be provided in the proximal portion to weight the handle appropriately.

The sterilising device 10 may be powered by mains electricity, by a rechargeable or non-rechargeable battery, a supercapacitor, or the like. In examples including a rechargeable power source, a charging port 13 may be provided to allow for convenient charging of the sterilising device 10. In examples including a mains powered sterilising device 10, or a sterilising device 10 relying on any form of external power supply, a power-in port may be provided in place of the charging port 13. The charging port 13 or power-in port may be positioned on the end face of the proximal portion 11 so as to not interfere with use of the sterilising device 10 whilst connected to external power. In examples including a port, there may be provided a cover, couplable to the port, for when the port is not in use.

The exterior surface of the proximal portion 11 may include one or more buttons configured to operate the sterilising device 10. One such button may be a power button 14. Another (not shown) may be configured to switch the device between multiple preset modes which may vary the light intensity and exposure duration of the UV light sources 18.

The power button 14 is preferably positioned toward either end of the proximal portion 11 such that it is convenient to press when the user is holding the sterilising device 10 ready to use. Other buttons may include functions to pre-select an operating mode of the sterilising device 10, to release a cover (shown in FIG. 2), or to indicate the charge level of the battery. The power button 14 may be configured in a number of ways. For example, the power button 14 may be pressed once to active the UV light sources 18, and the pressed again to deactivate the UV light sources 18. Alternatively, the power button 14 may be required to be held in order for the UV light sources 18 to be activated. Alternatively, the power button 14 may be pressed once to activate the UV light sources 18, the pressing of the power button also starting an internal timer. Once the internal timer ends, the UV light sources 18 may be deactivated. In some examples, the UV sterilising device 10 may comprise a sensor which can detect the microbial load in or on the medium to be sterilised, and the UV light sources 18 may deactivate once the microbial load is below a predetermined value.

The proximal portion 11 may further include a light bar 16 to provide feedback to the user. For example, the light bar 16 may illuminate a distinct colour dependent on the charge level of the battery. It will be appreciated that the annular light bar depicted in the figures is an example, and any configuration for a light may be used. Further, a light may not be present at all. In addition to or alternatively, the sterilising device 10 may include a haptic motor and/or a speaker with associated circuitry to provide haptic feedback or audible feedback to the user relating to the charge level of the battery.

The distal portion 12 of the sterilising device may include a rod 17, a plurality of UV light sources 18, and a protective casing 19. The rod 17 may be coupled to or be integral with the proximal portion 11. More preferably, the rod 17 may be coupled to an end of the proximal portion 11 as shown in FIG. 1. The rod 17 may be a substantially solid mass, or may instead be hollow to allow for improved weighting of the sterilising device 10. A hollow rod 17 also allows more simple wiring of the UV light sources 18.

At least one, but preferably a plurality of UV light sources 18, may be coupled to the outer surface of the rod 17. The UV light sources 18 may protrude from the outer surface to increase coverage of the emitted light. Alternatively, the UV light sources 18 may be positioned within a respective depression in the outer surface of the rod 17. In examples employing a plurality of UV light sources 18, the UV light sources 18 may be spaced along the length of and/or around the diameter of the rod 17. In some examples, the spacing is substantially even. The UV light sources, whether there are one or more, serve to project UV light radially around at least part of, but preferably substantially all the distal portion 12. In examples utilising multiple rows and/or columns of UV light sources 18, each column and/or row may be offset from adjacent rows to provide a more even spread of UV light.

The UV light sources 18 are configured to transmit UV-C, UV-B, or UV-A light, i.e., light having a wavelength of 100 nm to 280 nm, 280 nm to 315 nm, and 315 nm to 400 nm, respectively. More specifically, the wavelength of emitted light is preferably 180 nm to 350 nm. Preferably, the wavelength is in the UV-C range, and more preferably at or close to 254 nm.

Preferably, the at least one UV light source 18 is an LED UV light source, however other UV light sources are envisaged.

The protective casing 19 is configured to protect the UV light sources 18 from impact. The protective casing 19 further serves to prevent any liquid that is being pasteurised from coming into direct contact with the UV light sources. Such contact may cause unwanted reactions to the liquid, such as the calcification of milk. The casing 19 may be coupled either to the proximal portion 11 of the sterilising device 10, or to the rod 17. The casing 19 preferably forms a liquid-tight seal so as to protect the UV light sources 18 from the liquid being pasteurised. The use of further gaskets or a sealing medium may be used in some examples.

The casing 19 is of a material having a high transmittance of UV light, which is to say that preferably only minimal amounts of the UV light from the UV light sources 18 is absorbed or reflected by the casing 19. The casing 19 is also preferably substantially transparent or translucent. Suitable materials may be found, for example, in many glass, quartz, or polymer materials.

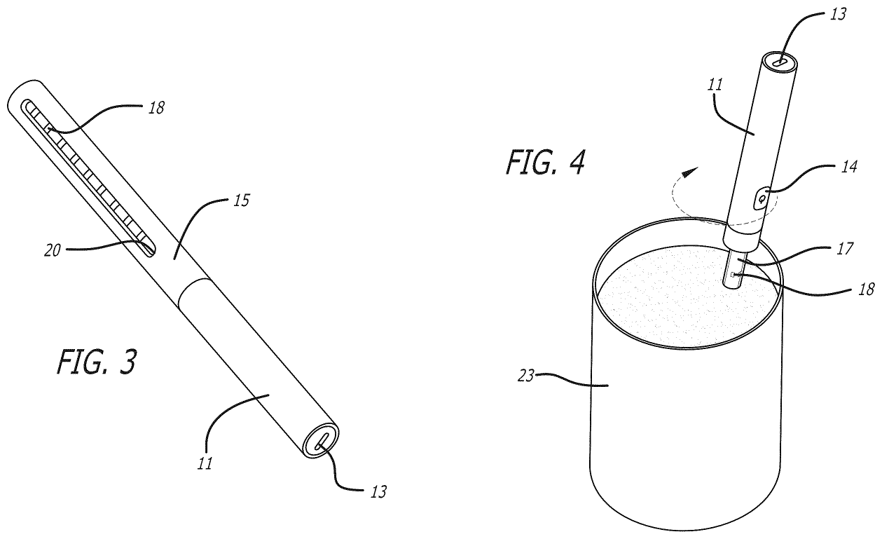

Referring to FIGS. 2 and 3, there may be provided a cover 15. The cover 15 serves to protect the distal portion 12, and specifically the protective casing 19, from damage which could reduce the efficacy of the UV light sources 18 acting on the medium to be sanitised. The cover 15 may couple to the casing 17 or proximal portion 11 by friction fit, threaded engagement, or any other coupling means. Preferably, the cover 15 is coupled to the proximal portion 11 by quick releasing coupling means for the convenience of the user.

In some examples, the cover 15 may include a slot 20. As illustrated in FIG. 3, the slot 20 may extend axially along the cover 15. Preferably, the slot 20 extends along at least the majority of the cover. The slot 20 may conveniently align with at least one of UV light sources 18. The cover 15 is of a material which absorbs and/or reflects UV light such that the UV light may only pass through the slot 20.

To ensure that at least one of the UV light sources 18 aligns with the slot 20, the proximal portion 11 and the cover 15 may include corresponding geometry, such as the projection 21 and indentation 22 as shown in FIG. 2.

Further, a sensor may be provided to recognise whether the cover 15 is in place. If the sensor recognises that the cover 15 is in place, the control unit 26 of the UV sterilising device 10 may cause only the row of UV light sources 18 adjacent the slot 20 to activate, whilst the other rows of UV light sources 18 remain deactivated. Similarly, if the sensor recognises that the cover is not in place, all UV light sources may be activated.

In some examples, the control unit 26 may deactivate the UV light sources 18 if the cover 15 is removed after the UV sterilising device 10 has been turned on by the user. This acts as a safety mechanism should the cover 15 be removed before the sanitisation of an object be complete.

FIG. 4 illustrates the UV sterilising device 10 being used, according to some examples, to pasteurise a liquid. Any liquid may be pasteurised by the UV sterilising device 10, whether the liquid be substantially transparent or opaque. Some non-limiting examples of suitable liquids are water, milk, and fruit juice. Similarly, the UV sterilising device 10 is preferably of materials capable of withstanding liquids at temperatures exceeding at least 80, at least 90, or at least 100 degrees Celsius without distortion or damage, and so can be used for hot beverages as well as cold beverages.

To use the UV sterilising device 10 to pasteurise a liquid, the cover 15 is preferably removed (in examples including the cover 15). The user then inserts at least part of the distal portion 12 into the vessel 23 holding the liquid, thereby submerging at least one of the UV light sources 18 in the liquid. In some examples, the distal portion 12 of the UV sterilising device 10 may be longer than the depth of the liquid in the vessel 23.

In order to minimise user exposure to UV radiation, the UV sterilising device 10 may comprise a depth sensor. The depth sensor may be configured to establish the liquid level relative to the distal portion 12, and only activate the UV light sources 18 which are surrounded by liquid. Further, the control unit 26 may automatically deactivate all, or at least some, of UV light sources 18 if the depth sensor detects that the user is removing the distal portion 12 from the liquid.

The efficacy of pasteurisation may be increased by the user using the distal end 12 of the UV sterilising device 10 to stir the liquid whilst the UV light sources are activated. Stirring the liquid is particularly advantageous when the liquid has a high opacity as more of the liquid is subject to UV radiation.

As referenced above, the UV sterilising device 10 may be capable of both pasteurising liquids and sanitising solids. Such solids may include foods, laptops, mobile phones, keyboards, work surfaces, or any other object. The control unit of the sterilising device 10 may not allow any UV light sources 18 to be activated without the cover 15 being in place, unless the distal end 12 is at least partially submerged in a liquid. Therefore, to sanitise a solid object, the cover 15 may need to be in place. Once the cover 15 is in place, the user may press the power button 14 and move the UV sterilising device 10 and the object to be sanitised relative to one another.

If the UV light sources 18 cannot be activated for any reason, the control unit may send a signal to at least one of a haptic motor, speaker, or the light bar 16. The user may therefore be alerted to the fact that the UV sterilising device cannot be used in its current condition. Various other alerts may be provided to the user, such as that the UV light sources 18 are activated, that the UV light sources 18 have been activated for a predetermined period of time, that the UV light sources 18 have been deactivated, or that the intensity of the UV light sources 18 has changed.

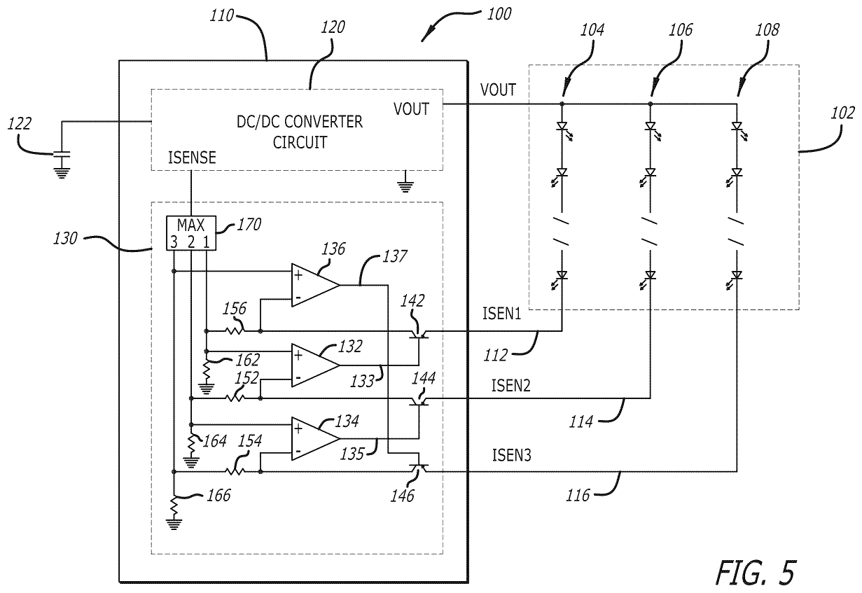

FIG. 5 illustrates one example of a known LED driver system 100. The system 100 may generally include an LED array 102 and LED controller circuitry 110. The LED array 102 includes a plurality of LED strings 104, 106 and 108. Each string 104, 106, and 108 may include a plurality of serially connected LEDs, for example, a first string 104 may include a plurality of LEDs connected in series, e.g., LED_11, LED_12 . . . , LED_ 1 n. In examples of this disclosure, the LEDs emit light in the UV spectrum, and preferably in the UV-C spectrum. Similarly, a second string 106 may include a plurality of LEDs connected in series, e.g., LED_21, LED_22 . . . , LED_2 n, and a third string 108 may include a plurality of LEDs connected in series, e.g., LED_31, LED_32 . . . , LED_3 n. Strings 104, 106 and 108 may be coupled together in parallel and to power supply, designated as Vout in the Figure. Thus, the voltage across each string may be represented by Vout. Each string may generate respective feedback signals 112, 114 and 116 (labelled Isen 1, Isen 2 and Isen 3, respectively). Feedback signals 112, 114 and 116 may be proportional to the current in each respective string.

LED controller circuitry 110 may include DC/DC converter circuitry 120 capable of generating a DC power Vout from a DC input 122. Controller circuitry 110 may individually or collectively comprise one or more integrated circuits. As used in any embodiment herein, an “integrated circuit” means a semiconductor device and/or microelectronic device, such as, for example, a semiconductor integrated circuit chip. Exemplary DC/DC converter circuitry 110 may include Buck, Boost, Buck-Boost, Sepic, Zeta, Cuk and/or other known or after-developed circuit topologies. Controller circuitry 110 may also include feedback circuitry 130 capable of balancing the current in each string of LEDs. In one embodiment, feedback circuitry 130 may be capable of comparing the current in one string to the current in at least one other string. The voltage drop of one or the other strings may be adjusted to adjust the current in one of the strings, based upon, at least in part, a difference between the relative current in the two LED strings. Exemplary operations of feedback circuitry 130 are discussed in greater detail below.

Feedback circuitry 130 may include amplifier circuitry 132, 134 and 136, one for each string 104, 106 and 108. Feedback circuitry may also include switches 142, 144 and 146, which may be configured to conduct respective feedback signals 112, 114 and 116. To that end, switches 142, 144 and 146 may be controlled such that the voltage drop across each switch may generate a desired current condition in each string of LEDs, as will be described herein. In this embodiment, switches 142, 144 and 146 may each comprise bipolar junction transistors (BJTs), where each respective current feedback signal 112, 11 and 116 is conducted from the emitter through the collector, and the base is controlled to control the value of the signal transmitted through the switch. Offset resistors 152, 154 and 156 may be coupled to each input of the amplifiers to reduce or eliminate offset errors which may be associated with the amplifiers. Sense resistors 162, 164 and 166 may be coupled to each respective current feedback signal 112, 114 and 116, and the input of each amplifier may be a voltage signal taken across respective sense resistors 162, 164 and 166. Sense resistors may be used to generate a proportional value of the feedback signals 112, 114 and 116. To achieve substantially equal current in each string of LEDs, the sense resistors may be substantially identical. However, and as will be described in embodiments below, the sense resistors may be selected to achieve different current values for each string of LEDs, relative to one another.

The current in any string may be proportional to Vout minus the voltage drop across an associated switch. Thus, for example, the current in string 104 may be proportional to Vout minus V (switch 142). Thus, by controlling the voltage drop across switch 142, the current in string 104 may be controlled. In this embodiment, the current in string 104 may be controlled relative to the current in string 106 by controlling the voltage drop across switch 142.

For example, in this embodiment, amplifier 132 may be configured to receive current feedback signal 112 (from the first string 104) via switch 142 and current feedback signal 114 (from the second string 106) via switch 144. More particularly, amplifier 132 may be configured to receive, at a non-inverting input, a voltage signal proportional to the current feedback signal 112 (taken across sense resistor 162) and, at an inverting input, a voltage signal proportional to the current feedback signal 114 (taken across sense resistor 164). Amplifier 132 may compare the relative values of signals 112 and 114 and generate a control signal 133. Control signal 133 may have a value that is based on, at least in part, the difference between signal 112 and 114. In this example, feedback current signal 112 may be applied to a non-inverting input of amplifier 132, and signal 114 may be applied to an inverting input of amplifier 132. Control signal 133 may control the conduction state of switch 142, for example, by controlling the base voltage of the switch 142. Each switch may be configured so that when balanced current flows through each string of LEDs, the output of the amplifier is at low state so that the switches are fully saturated. This may operate to reduce power losses associated with the transistors under such condition.

Controlling the conduction of switch 142 may operate to control the voltage drop across switch 142. As an example, if signal 112 is greater than signal 114, amplifier 132 may generate a higher control signal 133 (as compared to a state when signal 112 is equal to or less than signal 114). A higher control signal 133, applied to switch 142, may cause the base current to decrease and thus, the voltage drop across switch 142 to increase. Increasing the voltage drop across switch 142 may decrease the current 112 through LED string 104. This process may continue until the current values 112 and 114 are substantially identical. These operations illustrate the voltage drop across LEDs in string 104 has lower voltage drop than that of the voltage drop across LEDs in string 106.

Similarly, if signal 112 is less than signal 114, amplifier 132 may generate a lower control signal 133 (as compared to a state when signal 112 is equal to or greater than signal 114). A lower control signal 133, applied to switch 142, may cause the base current to increase and thus, the voltage drop across switch 142 to decrease. Decreasing the voltage drop across switch 142 may increase the current 112 through LED string 104. This process may continue until the current values 112 and 114 are substantially identical.

Amplifier 136 may be configured to receive current feedback signal 116 (from the third string 108) via switch 146 and current feedback signal 112 (from the first string 104) via switch 142. Amplifier 136 may compare the relative values of signals 116 and 112 and generate a control signal 137. Control signal 137 may have a value that is based on, at least in part, the difference between signal 116 and 112. In this example, feedback current signal 116 via sense resistor 166 may be applied to a non-inverting input of amplifier 136, and signal 112 via sense resistors 156, 162 may be applied to an inverting input of amplifier 136. Control signal 137 may control the conduction state of switch 146, for example, by controlling the base voltage of the switch 146. Controlling the conduction of switch 146 may operate to control the voltage drop across switch 146. As an example, if signal 116 is greater than signal 112, amplifier 136 may generate a higher control signal 137 (as compared to a state when signal 116 is equal to or less than signal 112). A higher control signal 137, applied to switch 146, may cause the base current to decrease and thus, voltage drop across switch 146 to increase. Increasing the voltage drop across switch 146 may decrease the current 116 through LED string 108. This process may continue until the current values 116 and 112 are substantially identical.

Similarly, if signal 116 is less than signal 112, amplifier 136 may generate a lower control signal 137 (as compared to a state when signal 116 is equal to or greater than signal 112). A lower control signal 137, applied to switch 146, may cause the voltage drop across switch 146 to decrease. Decreasing the voltage drop across switch 146 may increase the current 116 through LED string 108. This process may continue until the current values 116 and 112 are substantially identical.

In this embodiment, feedback signal 112, 114 and/or 116 may be supplied to DC/DC converter circuitry 120. Based upon, at least in part, the value of feedback signal 112, 114 and/or 116, DC/DC converter circuitry 120 may be capable of adjusting Vout to achieve preset and/or desired current conditions in at least one LED string 104, 106 and/or 108. Although not shown in this figure, it is equally contemplated under this embodiment that controller circuitry 110 includes user-controllable circuitry (which may comprise, for example, software and/or hardware) to preset a desired brightness of the LCD panel. In that instance, DC/DC converter circuitry may adjust power to the LED array based on the preset value as set by the user and the value of feedback signal 116.

Feedback circuitry 130 may also include pass-through circuitry 170 capable of providing at least one feedback signal 112, 114 and/or 116 to the DC/DC converter circuitry 120. In this embodiment, pass-through circuitry may operate as an OR gate, allowing at least one of the feedback signals across sense resistor 162, 164 and/or 166 to flow through to converter circuitry 120. This may enable, for example, circuitry 120 to continue to receive feedback information in the event that one or more strings 104, 106 and/or 108 becomes an open circuit.

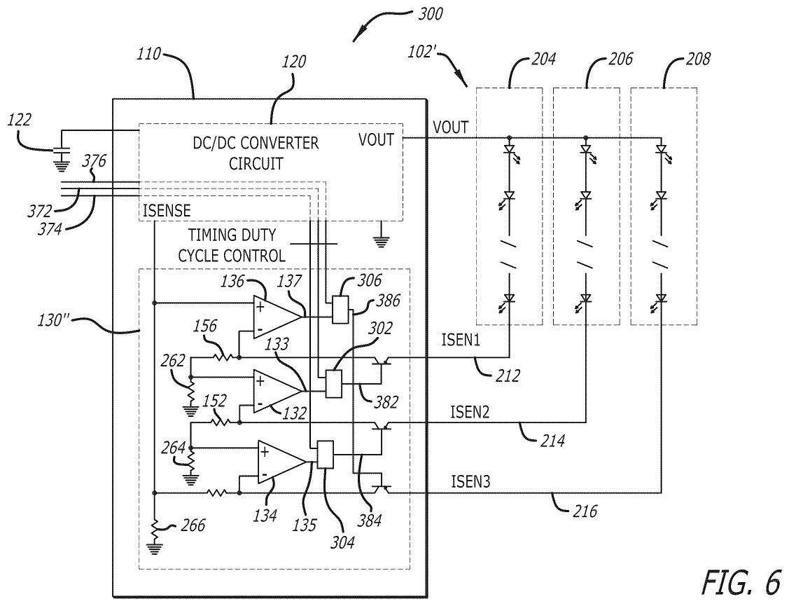

FIG. 6 illustrates another example of a known LED driver system 300. In this example, feedback circuitry 130″ may include burst mode dimming circuitry which may control the brightness of at least one LED string 204, 206 and/or 208. Burst mode dimming circuitry may be capable of adjusting the brightness of string 204, 206 and/or 208 by regulating the flow of the feedback signal 212, 214 and/or 216, as will be described below.

Feedback circuitry 130″ may include multiplexer circuitry 302, 304 and 306. Multiplexer 302 may have a first input configured to receive a pulse width modulated (PWM) signal 372 and a second input configured to receive control signal 133. The multiplexer circuitry 302 may generate an output signal 382 based on the PWM signal 372 and control signal 133. The PWM signal 372 may comprise a low frequency burst mode signal, and may be designated for specific brightness control of the LED string 204. For example, the PWM signal 372 may comprise a rectangular waveform having a selected ON-OFF duty cycle, i.e., the waveform swings from HIGH to LOW based on a selected duty cycle. The frequency of the PWM signal 372 may be selected to avoid flickering of the LEDs, for example, several hundred Hertz.

In operation, if the PWM signal 372 is HIGH, the output signal 382 of the multiplexer may be the control signal 133. Thus, when the PWM signal 372 is HIGH, switch 142 may be controlled by control signal 133 in a manner described above. If the PWM signal 372 is LOW, the output signal 382 may be driven HIGH so that the switch 142 is turned OFF. Of course, the output signal 382 may be driven HIGH when the PWM signal is LOW by simply reversing the logic inside the multiplexer. In this case, the LED string 204 may be an open circuit and no current may flow through the LEDs. In this manner, LED string 204 may be repeatedly turned ON and OFF at a selected duty cycle to adjust the average current flow through the string 204 for performing the dimming control, which may to achieve a desired brightness of string 204.

Multiplexer 304 may have a first input configured to receive a pulse width modulated (PWM) signal 374 and a second input configured to receive control signal 135. The multiplexer circuitry 304 may generate an output signal 384 based on the PWM signal 374 and control signal 135. The PWM signal 374 may comprise a low frequency burst mode signal, and may be designated for specific brightness control of the LED string 206. For example, the PWM signal 374 may comprise a rectangular waveform having a selected ON-OFF duty cycle, i.e., the waveform swings from HIGH to LOW based on a selected duty cycle. The frequency of the PWM signal 374 may be selected to avoid flickering of the LEDs, for example, several hundred Hertz.

In operation, if the PWM signal 374 is HIGH, the output signal 384 of the multiplexer may be the control signal 135. Thus, when the PWM signal 374 is HIGH, switch 144 may be controlled by control signal 135 in a manner described above. If the PWM signal 374 is LOW, the output signal 384 may be driven HIGH so that the switch 144 is turned OFF. Of course, the output signal 384 may be driven HIGH when the PWM signal is LOW by simply reversing the logic inside the multiplexer. In this case, the LED string 206 may be an open circuit and no current may flow through the LEDs. In this manner, LED string 206 may be repeatedly turned ON and OFF at a selected duty cycle to adjust the average current flow through the string 206, which may achieve a desired brightness of string 206.

Multiplexer 306 may have a first input configured to receive a pulse width modulated (PWM) signal 376 and a second input configured to receive control signal 137. The multiplexer circuitry 306 may generate an output signal 386 based on the PWM signal 376 and control signal 137. The PWM signal 376 may comprise a low frequency burst mode signal, and may be designated for specific brightness control of the LED string 208. For example, the PWM signal 376 may comprise a rectangular waveform having a selected ON-OFF duty cycle, i.e., the waveform swings from HIGH to LOW based on the selected duty cycle. The frequency of the PWM signal 376 may be selected to avoid flickering of the LEDs, for example, several hundred Hertz.

In operation, if the PWM signal 376 is HIGH, the output signal 386 of the multiplexer may be the control signal 137. Thus, when the PWM signal 376 is HIGH, switch 146 may be controlled by control signal 137 in a manner described above. If the PWM signal 376 is LOW, the output signal 386 may be driven HIGH so that the switch 146 is turned OFF. Of course, the multiplexer of this embodiment may be configured so that output signal 386 may be driven HIGH when the PWM signal is LOW. In this case, the LED string 208 may be an open circuit and no current may flow through the LEDs. In this manner, LED string 208 may be repeatedly turned ON and OFF at a selected duty cycle to adjust the average current flow through the string 208, which may achieve a desired brightness of string 208.

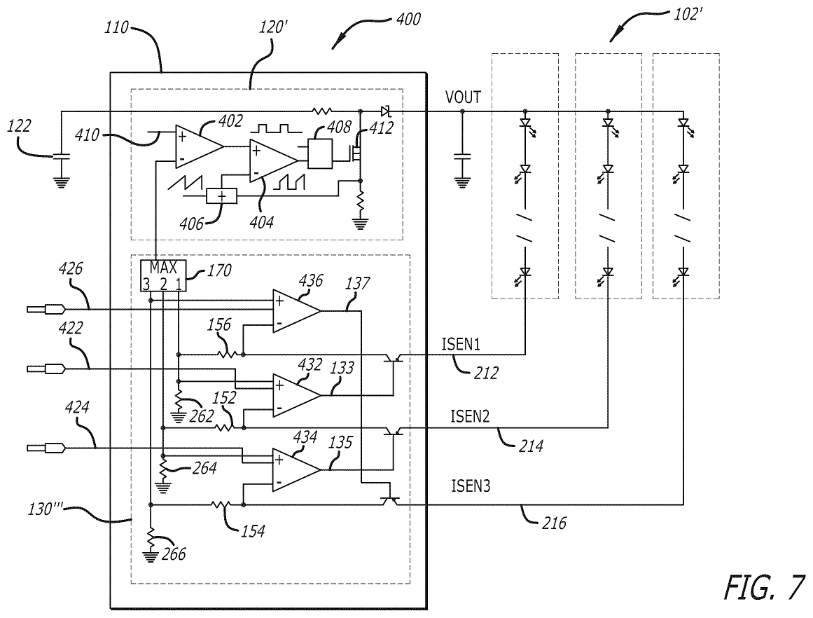

FIG. 7 illustrates another example of a known LED driver system 400. In this example, DC/DC converter circuitry 120′ may include a boost converter. The boost converter may include a first comparator 402 that compares one of the current feedback signals from the LED array 102′ to an adjustment signal. Error amplifier 402 compares the current sense signal Isen, and a reference signal ADJ. The result of the signal is comparing with a slope compensated current sense signal in the switch of the boost converter. The current flowing through switch is added with a saw-tooth via 406. The output of the 406 is one of the inputs to comparator 404. The output of the comparator 404 is a rectangular wave which feeds into a driver such as a flip-flop, to drive switch in the boost converter.

As described above, the ratio of current flow through each string may be adjusted by burst mode dimming and/or by selecting the values of the sense resistors 262, 264 and/or 266. In this embodiment, feedback circuitry 130″ may include amplifiers 432, 434 and 436 which may be capable of adjusting the effective resistance of associated sense resistors 262, 264 and/or 266, respectively. In this example, programmable input signals 422, 424 and 426 may be supplied to respective amplifiers 432, 434 and 436. Programmable input signals 422, 424 and 426 may be proportional to a desired current level in a given string.

In operation, the value of input signal 422 may be adjusted up or down, and accordingly, the effective resistance of sense resistor 262 may be adjusted up or down. As described above, this may form a ratio of current values between the first and second strings. The value of input signal 424 of may be adjusted up or down, and accordingly, the effective resistance of sense resistor 264 may be adjusted up or down. As described above, this may form a ratio of current values between the second and third strings. Similarly, the value of input signal 426 of may be adjusted up or down, and accordingly, the effective resistance of sense resistor 266 may be adjusted up or down. As described above, this may form a ratio of current values between the third and first strings. These operations may produce a desired and/or programmable current flow through one or more LED strings.

Any of the examples described herein may be extended to include n-number of LED strings. In accordance with the teachings herein, if n-number of LED strings are used, a corresponding number of amplifier circuits and switches may also be used. Likewise, a corresponding number of multiplexer circuits may be used, depending on the number of LED strings present.

In some examples of this disclosure, the device comprises a processor and a memory, the memory storing executable instructions which, when executed by the processor, cause the processor to perform at least one function of the device. In some examples, the memory stores data indicative of the operation of the device by a user, such as the duration and/or intensity of the UV light emitted from the device.

In some examples of this disclosure, the device comprises a wireless communication system, preferably in the form of a Bluetooth Low Energy capable microcontroller. The wireless communication system is in communication with the processor of the device and is configured to transmit and receive data between the driver device and a computing device, such as a smartphone. The device may therefore transmit data to a computing device which in turn transmits the data to the cloud or a remote server for processing.

The connectivity via Bluetooth Low Energy to a companion mobile application ensures that only small power for this communication is required thus allowing the device to remain functioning for a longer period of time if not used at all, compared to traditional wireless connectivity solutions like Wi-Fi or classic Bluetooth. However, in other examples, the device is configured to communication with a computing device using Wi-Fi or classic Bluetooth.

It will be appreciated that various examples above may produce heat as a byproduct of using the UV sterilising device 10, and specifically by activating the UV light sources 18. The UV sterilising device may therefore implement an integrated cooling system such that the power source and circuitry remain at an appropriate temperature. An integrated cooling system may include at least one of the group comprising a heat sink, an air vent, and a fan. A plurality of heat sinks, air vents, and/or fans may be implemented as appropriate. Of course, in some examples cooling may not be required, or components of the UV sterilising device 10 itself may serve as a heat sink. In further examples, a docking station may be provided for the UV sterilising device which implements a cooling system between uses of the UV sterilising device 10.

### Artificial Intelligence Control

Aspects of the disclosure provide apparatus, systems and methods that may be at least partly implemented using machine learning (ML) and/or artificial intelligence (AI).

Machine learning and artificial intelligence techniques may be implemented in hardware, software, firmware, or any combination thereof. Trained machine learning models (ML models) may be implemented as hardware, or computer programs running on conventional or specialised hardware. Trained ML models may be implemented by one or more local processing devices (e.g. client-side devices) and/or one or more remote processing devices (e.g. server-side devices). Computer programs for implementing ML models may be stored in any suitable storage medium such as random access memory (RAM), read only memory (ROM) flash memory, a hard disk and so on.

Aspects of the disclosure may use one or more trained ML models which may be suitably trained for the purposes of any of the techniques disclosed herein. Any of the techniques disclosed herein may be implemented using one or more ML models trained using one or more of supervised learning, unsupervised learning, semi supervised learning and reinforcement learning.

Trained ML models may be implemented as processor-implemented artificial neural networks (ANNs) using one or more of: one or more central processing units (CPUs), one or more graphics processing units (GPUs), one or more digital signal processors (DSPs), one or more field programmable gate arrays (FPGAs), one or more application specific integrated circuits (ASICs), and one or more deep learning processors (DLP).

More generally, aspects of the disclosure may provide one or more processing devices and/or one or more processing systems operable to execute one or more trained ML models to receive an input (e.g. an appropriate feature vector) and map the input to an output according to a learned function. Any of the processing devices and systems discussed herein may be operable to execute such an ML model for performing the task(s) and purpose(s) described in relation to that processing device or system.

An artificial neural network comprises layers of interconnected artificial neurons. These layers can be grouped as an input layer, one or more hidden layers, and an output layer. An input (e.g. an appropriate feature vector) is provided to the input layer and processed by the layers of the artificial neural network according to learned parameters (e.g. weights and biases) to obtain an output at the output layer. Processes to learn these parameters are typically carried out in advance as part of a training stage.

Aspects of the disclosure may use any suitable artificial neural network architecture. Examples of suitable architectures include feed forward neural networks (FFN), convolutional neural networks (CNN), recurrent neural networks (RNN), and generative adversarial networks (GAN) among others.

ML model training and inference stages may be implemented using any suitable combination of hardware components. In some examples, ML model training may use one or more GPUs, whereas ML model inference may use one or more CPUs.

Supervised learning techniques using labelled training data may be used to train ML models to learn a function for mapping an input to an output. The labelled training data (and indeed the inputs during inference) may correspond to various data types, such as numerical values, text, image data, video data, audio data and so on. Using labelled examples, the ML model can map and input to an output. A loss function allows for updating of learned parameters to minimise differences (loss) between ML model outputs and ground truths represented by the labels. More generally, backpropagation algorithms may be used for optimising learned parameters. In some examples, supervised learning techniques may be used for tasks such as classification, regression and forecasting.

Semi supervised learning techniques using both labelled training data and unlabelled training data may be used to train ML models to learn a function for mapping an input to an output. In some examples, semi-supervised support vector machines may be used for implementing semi supervised learning techniques.

Various types of unsupervised learning techniques trained on unlabelled data may be used for tasks such as clustering (e.g. k-means clustering), association, and dimensionality reduction. In some examples, one or more of an autoencoder, variational autoencoder (VAE), convolutional variational autoencoder (CVAE), and generative adversarial network (GAN) may be used. Network architectures for autoencoders and GANs are generally known and not discussed here.

Aspects of the disclosure may be implemented using reinforcement learning techniques. Examples of suitable reinforcement learning algorithms may include Q-learning, deep Q-networks (DQNs), SARSA (state-action-reward-state-action), or deep deterministic policy gradient (DDPG). AI agents can be trained according to reinforcement learning techniques to learn a policy for interacting with an environment to perform actions for maximising a cumulative reward.

Aspects of the disclosure may use so-called generative AI models which may typically use unsupervised or semi-supervised machine learning techniques. The generative AI models learn the patterns and structure of input training data and generate new data having similar characteristics. Examples of suitable generative AI models that may be used include autoencoders, GANs, auto-regressive neural networks, and transformer-based deep neural networks. Any suitable generative AI model may be used in combination with the techniques disclosed herein.

More generally, any of the above-mentioned ML and AI techniques may be suitably combined with any of the techniques disclosed herein for providing further technical improvements.

In embodiments of the disclosure, the control unit is configured to use at least one machine learning (ML) model to control at least one of the exposure duration and the UV light intensity. References to the control unit using at least one ML model may refer to any of an ML model that may be run locally by the control unit (e.g. without communication with an external device), an ML model that may be run by an external processing device (e.g. a mobile user device, such as a smartphone device or similar) that communicates with the portable UV sterilising device via a short-range wireless communication (e.g. NFC, Bluetooth®, ZigBee®), and/or an ML model that may be run by a remote server (e.g. cloud sever) which may be accessible to the control unit via the external processing device (e.g. smartphone device).

In embodiments of the disclosure, the portable UV sterilising device comprises the control unit (e.g. comprising one or more CPUs and associated memory). The control unit may for example comprise one or more of a System-on-Chip (SoC), FPGA, ASIC, microcontroller and so on for performing the functionality described in relation to the control unit. The control unit can be operable to control at least one of an exposure duration and UV light intensity for the UV light sources.

As explained previously, the portable UV sterilising device 10 comprises a plurality of UV light sources 18. The plurality of UV light sources 18 may comprise a plurality of strings, in which each string may comprise one or more UV light sources 18. References to controlling the UV light sources 18 may refer to any of controlling all of the UV light sources 18 (i.e. a same control applied for each of the UV light sources 18), or controlling individual strings (or more generally controlling subsets of the UV light sources 18) or controlling one or more individual UV light sources 18. In some cases, the control unit may control exposure duration and/or UV light intensity so that this is the same for each of the UV light sources 18. In some cases, the control unit may control UV light intensity for a first subset of the UV light sources. For example, when activated, the portable UV sterilising device may operate with a first subset of the UV light sources operating according to a default light intensity and a second subset of the UV light sources operating according to a controlled light intensity.

In some embodiments of the disclosure, the portable UV sterilising device may comprise N UV light sources, where N may be any suitable value that is two or more. In some examples, N may be a value in the range 5-100. The portable UV sterilising device 10 comprises the control unit which may control UV light intensity and/or exposure duration for each of the N UV light sources 18. A light intensity can be controlled (e.g. modified) by controlling (e.g. modifying) an electrical current supplied to an LED (e.g. LED string). An exposure duration can be controlled by enabling and disabling supply of electrical current for an LED (e.g. LED string). References to exposure duration for an LED refer to a length of time that the LED is in active state and emitting light (and thus providing UV radiation to a nearby subject within range).

In some examples, the portable UV sterilising device 10 may operate using a predetermined exposure duration for a treatment session. For example, a predetermined exposure duration of the order of tens of seconds or minutes may be specified. In such cases, using the at least one ML model, the control unit may control UV light intensity during the predetermined exposure duration to provide an appropriate amount of UV treatment for a subject. For a larger desired treatment effect, the LEDs can be controlled to provide a greater UV light intensity during the treatment session. Conversely, for a smaller desired treatment effect, the LEDs can be controlled to provide a smaller UV light intensity during the treatment session. In some examples, using the at least one ML model, the control unit may select an exposure duration from a plurality of predetermined exposure durations, and also control the UV light intensity during the exposure duration so that the combination of the two provides an appropriate amount of UV treatment for a subject. For example, a plurality of predetermined exposure durations may be specified each corresponding to a different duration (e.g. each Y second increments between each of the predetermined exposure durations, where Y may be any suitable value such as 30 seconds).

In some examples, the portable UV sterilising device 10 may operate using a predetermined UV light intensity for a treatment session. In such cases, using the at least one ML model, the control unit may control exposure duration to provide an appropriate amount of UV treatment for a subject. For a larger desired treatment effect, the LEDs can be controlled to provide a longer treatment session. Conversely, for a smaller desired treatment effect, the LEDs can be controlled to provide a shorter treatment session. Hence, in some examples treatment session duration may be controlled while using a fixed UV light intensity.

In some examples, both the exposure duration and the UV light intensity may be controlled to provide an appropriate amount of UV treatment for a subject. In some examples, the control unit may be operable to use at least one ML model to control each of exposure duration and UV light intensity according to a continuous scale (e.g. any suitable exposure duration and any suitable light intensity within the LED capability). Alternatively or in addition, the portable UV sterilising device 10 may have a plurality of selectable modes of operation (which may be user-selectable and/or automatically selectable) which may have different predefined exposure durations and different predefined UV light intensities. Using the at least one ML model, the control unit may be operable to automatically control the portable UV sterilising device to operate according to a respective mode of the plurality of selectable modes so as to provide an appropriate amount of UV treatment for a subject.

In some examples, the plurality of selectable modes of operation may comprise two or more first exposure modes each having a first exposure duration and different UV intensities so that the two or more first exposure modes are each capable of providing a different amount of UV treatment over the first exposure duration. The plurality of selectable modes of operation may further comprise two or more second exposure modes each having a second exposure duration and different UV intensities so that the two or more second exposure modes are each capable of providing a different amount of UV treatment over the second exposure duration. In a similar manner, there may be two or more third exposure modes, two or more fourth exposure modes and so on. More generally, exposure duration and the UV light intensity may be controlled based on selection of a respective one of the plurality of selectable modes of operation.

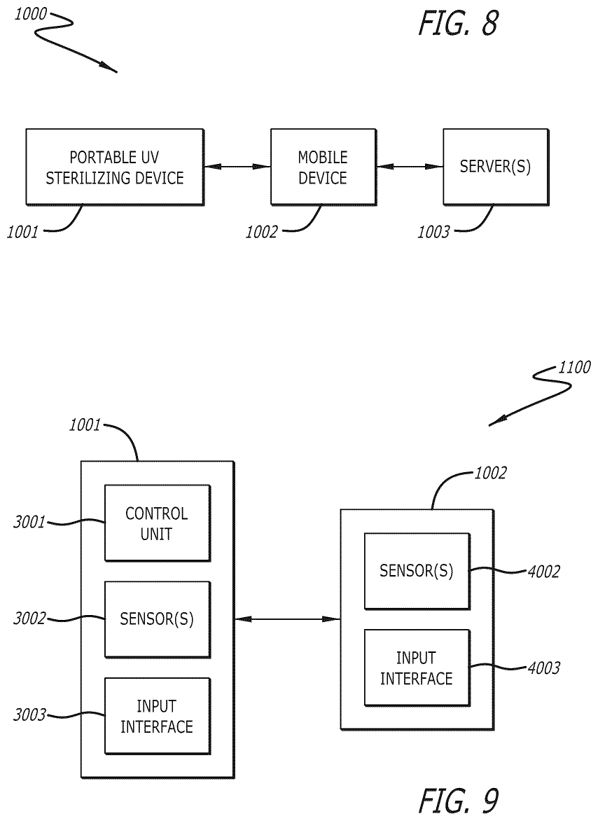

FIG. 8 schematically illustrates an example of a system 1000. The system 1000 comprises the portable UV sterilising device 1001, a mobile user device 1002 and optionally one or more servers 1003. The arrows in FIG. 8 indicate communications between these devices. The portable UV sterilising device 1001 and the mobile user device 1002 may communicate using any suitable short-range network (e.g. Bluetooth®). The mobile user device 1002 and the one or more servers 1003 may communicate using various communication networks (e.g. local area networks, mobile networks, the Internet). In some examples, the mobile user device 1002 may connect to a mobile network and/or a Local Area Network (LAN) and may access the one or more servers via Wide Area Networks (WANs), such as the Internet. FIG. 8 thus represents an example in which any of the portable UV sterilising device 1001, mobile user device 1002, and one or more servers 1003 may run one or more ML models for controlling the portable UV sterilising device 1001.

In some arrangements, the machine learning techniques of the present disclosure may be performed using the portable UV sterilising device 1001 as a standalone device. In other arrangements, the machine learning techniques of the present disclosure may be performed using the portable UV sterilising device 1001 together with the mobile user device 1002. The mobile user device 1002 may detect properties for a subject to be treated, or being treated, (e.g. subject type, subject quantity, microbial load) using sensors such as image sensors and/or audio sensors. For example, an image may be captured and analysed (e.g. using traditional image processing techniques and/or computer vision techniques) to detect object types, and object quantity (e.g. volume). In some examples, microbial load may be detected based on an image comprising a date (e.g. sell-by date or best-before date). Moreover, based on a sell-by date, a microbial load may be estimated (e.g. in units of per gram or per ml). The above examples refer to image analysis. Alternatively or in addition, an audio sensor may be provided (e.g. included in the portable UV sterilising device 1001 and/or the mobile user device 1002) for detecting speech input. A user may provide speech input to specify a subject type, subject quantity and microbial load. For example, a user may provide speech input specifying a sell-by date, or potentially a rating (e.g. high, low, medium) for a relative microbial load that may be known or perceived by the user. Any of these inputs can be provided to the ML model. Any of the above-mentioned sensors may be provided as part of the portable UV sterilising device 1001 and/or the mobile user device 1002. In some examples, the mobile user device 1002 may be a smartwatch device or smartphone device and any sensors typically associated with known smartwatches and smartphones may be used.

Hence more generally, the control unit is configured to use at least one ML model which may be local or remote with respect to the control unit. Generally speaking, the ML model has been trained in advance for performing control of the portable UV sterilising device (specifically control of LEDs to control light intensity and/or exposure duration). Data for learned parameters (e.g. weights, biases) and the model architecture can be loaded to any of devices discussed above, and any of the input data discussed herein can be provided as input to the ML model for inference to obtain outputs for providing control of the LEDs. More generally, input data can be fed to the ML model, and using the set of learned parameters, the ML model can provide one or more outputs based on the input data and the set of learned parameters, in which the outputs allow the control unit to control the LEDs. References to the ML model controlling least one of the exposure duration and the UV light intensity refer to the ML model providing outputs which are used by the control unit (e.g. automatically instruct the control unit) to automatically control the LEDs (e.g. by varying supplied current and/or starting/stopping supply of current).

In some embodiments of the disclosure, the at least one ML model may be operable to control at least one of the exposure duration and the UV light intensity in dependence on input data indicative of at least one of: a type of subject to be treated, a quantity of the subject, and an initial microbial load associated with the subject. The portable UV sterilising device may comprise one or more sensors (e.g. audio sensors and/or image sensors) operable to detect at least some of the input data. Alternatively or in addition, the portable UV sterilising device may comprise an input interface (e.g. one or more buttons, a display unit, touchscreen display) operable to receive user input for specifying at least some of the input data. Alternatively or in addition, the portable UV sterilising device may comprise wireless communication circuitry operable to receive at least some of the input data from a mobile user device (e.g. 1002) via a wireless communications network.

FIG. 9 schematically illustrates an example of a system 1100 comprising the portable UV sterilising device 1001 and the mobile device 1002. In this example, the portable UV sterilising device 1001 comprises the control unit 3001, one or more sensors 3002, and an input interface 3002. The mobile device 1002 comprises one or more sensors 4002 and an input interface 4003. It will be appreciated that for arrangements in which the portable UV sterilising device 1001 comprises the one or more sensors 3002 and the input interface 3003, the mobile device 1002 may not be present. Conversely, it will be appreciated that for arrangements in which the portable UV sterilising device 1001 is used with the mobile device 1002 (e.g. a smartphone device), the portable UV sterilising device 1001 may not comprise the one or more sensors 3002 or the input interface 3002.

Still referring to FIG. 9, the input interface 4003 may comprise a touchscreen (e.g. as found on typical smartphone devices, tablet devices, smartwatch devices and other similar devices). The mobile device 1002 may be operable to run a software application (e.g. mobile application) associated with the portable UV sterilising device 1001 and may display a graphical user interface (GUI) or similar for allowing a user to provide input data. API techniques may be used for transfer of data between the mobile device 1002 and the portable UV sterilising device 1001. The sensor(s) 4002 may for example correspond to a camera and/or a microphone as commonly found in existing smartphone devices.

Hence more generally, input data obtained by one or both of the portable UV sterilising device 1001 and the mobile device 1002 can be input to an ML model for allowing control of the portable UV sterilising device 1001 responsive to one or more current properties associated with a current subject that is to be treated (or being treated). The ML model may control the portable UV sterilising device 1001 responsive to one or more of: type of subject to be treated, a quantity of the subject to be treated, and an initial microbial load associated with the subject to be treated. Using ML to control the portable UV sterilising device 1001 responsive to one or more of these properties can allow appropriate UV treatment for a subject for reliable sterilisation of a subject. In addition, efficient operation of the portable UV sterilising device can be achieved which may prolong battery life for battery powered arrangements and useful lifespan.

In some examples, the portable UV sterilising device 1001 may comprise wireless communication circuitry to receive at least some of the input data from a mobile user device (e.g. 1002) and then provide the input data to an ML model locally run on the portable UV sterilising device 1001. Alternatively, the wireless communication circuitry may receive one or more control signals for controlling at least one of the exposure duration and the UV light intensity, in which the one or more control signals may be based on outputs of an ML model, run by mobile device 1002, in response to input data obtained by the mobile device 100. As already mentioned, in some examples an ML model may be run by one or more remote servers (e.g. accessible via the Internet).

For different food types and/or different beverage types, the ML model may control the portable UV sterilising device to provide different UV light intensity and/or different exposure durations. Some food types and/or beverage types may be capable of treatment using lower UV light intensity and/or shorter exposure duration to reduce microbial load to an acceptable level for that food type or beverage type, whereas other food types and/or beverage types may require treatment using higher UV light intensity and/or longer exposure duration to reduce microbial load to an acceptable level for that food type or beverage type. Moreover, properties such as density and opacity may influence how the subject is to be treated. For example, greater UV light intensity and/or greater exposure duration may be used for treatment of beverages with higher opacity and higher density, and conversely lower UV light intensity and/or shorter exposure duration may be used for beverages with lower opacity and lower density. Furthermore, an acceptable level of microbial load (e.g. in units of colony forming units (CFU) per gram) may vary for different food types/beverage types. For example, beverages such as milk (or more generally dairy-based beverages) may have acceptable levels that are lower than other beverages such as fruit juices. It will be appreciated that milk and fruit juice are examples of broad classifications and that classifications may be more granular (e.g. using sub-classes) with different types of milks and different types of fruit juices having different acceptable levels.

For different quantities of a same food type and/or different quantities of a same beverage type, at least one of a different UV light intensity and/or a different exposure duration can be used. Generally speaking, for a same type of subject (e.g. food, beverage), for a larger quantity the ML model can control the portable UV sterilising device to increase at least one of the UV light intensity and the exposure duration for treating that subject, and for a smaller quantity the ML model can control the portable UV sterilising device to decrease at least one of the UV light intensity and the exposure duration for treating that subject. More generally, for larger quantities a relatively larger UV light intensity and/or exposure duration can be used, whereas for smaller quantities a relatively smaller UV light intensity and/or exposure duration can be used.

For different microbial loads, at least one of a different UV light intensity and/or a different exposure duration can be used. For a larger microbial load for a given subject, the ML model can control the portable UV sterilising device to increase at least one of the UV light intensity and the exposure duration for treating the given subject, whereas for a smaller microbial load for another subject the ML model can control the portable UV sterilising device to decrease at least one of the UV light intensity and the exposure duration for treating the another subject. Moreover, for a same type of subject and having a same quantity, for a larger microbial load for that subject the ML model can control the portable UV sterilising device to increase at least one of the UV light intensity and the exposure duration for treating that subject, and for a smaller microbial load for that subject the ML model can control the portable UV sterilising device to decrease at least one of the UV light intensity and the exposure duration for treating that subject. For example, for two respective one litre samples of milk (e.g. cow's milk) having two different sell-by dates, a first treatment using at least one of a higher UV light intensity and/or a longer exposure duration can be used for the sample having the older sell-by date, and a second treatment using at least one of a lower UV light intensity and/or a shorter exposure duration can be used for the sample having the more recent sell-by date.

The above represents an example in which microbial load is estimated based on sell-by date (or other similar dates for indicating age of the subject) which may be input based on speech and/or a captured image and/or manual user input. In some examples, a sensor may be used to measure a microbial load and a measured microbial load may be used in the techniques of the present disclosure. For example, one or more of the portable UV sterilising device, the mobile device and/or a kit (e.g. a kit that may optionally be supplied with the portable UV sterilising device) may comprise one or more sensors for detecting microbial load. Alternatively or in addition, an input interface may receive a user input specifying a microbial load (e.g. in units of CFU per gram or litre). For example, a manual or webpage may be available to a user which includes information that can be referenced by the user for providing an estimated microbial load for a subject based on subject type and subject age (e.g. a current date relative to a sell-by date, use-by-date, best before date, or manufactured-on date). For example, one or more tables listing estimated microbial loads and relative ages may be reference by the user.

Hence more generally, in some embodiments of the disclosure input data indicative of at least one of a type of subject to be treated, a quantity of the subject to be treated, and an initial microbial load associated with the subject to be treated can be input to an ML model, in which the ML model has been trained to map the input to an output for controlling the control unit 3001 to control at least one of the exposure duration and the UV light intensity for the UV light sources 18. The UV light sources 18 can thus be automatically controlled for the subject that is to be treated to deliver an appropriate amount of UV treatment (by suitably controlling at least one of the UV light intensity and/or exposure duration) for the subject which can allow safe consumption of the treated subject by one or more end users (e.g. humans, pets or other animals) once treated. Furthermore, the UV light sources 18 can be automatically controlled to achieve efficient operation for the portable UV sterilising device for performing the treatment (e.g. by avoiding unnecessary over treatment, and potentially optimising power consumption for subject treatment).

ML model training may use any suitable machine learning training techniques. Any of supervised learning, unsupervised learning, semi-supervised learning and reinforcement learning may be used for training. In some examples, an ML model may have been trained (at least partly) using training data comprising the following attributes: subject type, subject quantity, initial microbial load before treatment, final microbial load after treatment, and properties of UV light used for treatment including UV light intensity and UV exposure duration. Training data relating to different subject types (e.g. food types, beverage types, object types) may be used. In some examples, the training data may further comprise acceptable levels of microbial load for different subject types. During training, parameters can be learned for mapping an input comprising properties of a subject to be treated (e.g. type, quantity, microbial load) to an output indicative of UV light intensity and/or UV exposure duration for successfully treating the subject to at least at or below an acceptable microbial load. In some examples, the training data may comprise measurements previously obtained when using another similar device having the same or similar LED technology as the portable UV sterilising device (e.g. a prototype or earlier version of the portable UV sterilising device). Supervised machine learning may be used to train at least one ML model to output classifications and/or regression values. Classifications may be output which may correspond to modes of operation for the portable UV sterilising device. More generally, classifications corresponding to different UV light intensity and different exposure durations may be output. Alternatively or in addition, regression values indicative of values for UV light intensity and/or exposure durations (e.g. values for supply currents for LEDs and durations) may be output. Examples of selectable modes of operation are discussed in more detail later.

In some embodiments of the disclosure, the portable UV sterilising device may comprise one or more batteries (which may or may not be rechargeable) for powering the UV light sources. The at least one ML model may be operable to control at least one of the exposure duration and the UV light intensity in dependence on input data indicative of a remaining battery charge (e.g. state of charge SOC) for one or more batteries for powering the portable UV sterilising device. The remaining battery charge may be determined using any suitable technique (e.g. current and/or voltage measurement). Techniques for determining remaining battery charge (e.g. SOC, where 100% corresponds to full) are generally known and are not discussed in detail.

In some embodiments of the disclosure, in response to the remaining battery charge falling below a preset level, control of the exposure duration and the UV light intensity may be performed to optimise power consumption. Generally speaking, power consumed by LEDs increases with increasing light intensity. Moreover, larger LED currents are typically associated with larger operating temperatures and reduced operational efficiency (and potentially junction heating can contribute to reduced lifespan). Furthermore, larger temperatures can also contribute to increased power consumption for cooling systems. In the techniques of the present disclosure, in response to the remaining battery charge falling below a preset level (e.g. 10%, 20%, 30% or other similar values), the control unit can be configured to use the at least one ML model to control the exposure duration and the UV light intensity to optimise power consumption for treatment of a subject. In other words, the exposure duration and the UV light intensity can be controlled to prioritise reducing energy usage while maintaining desired functionality (i.e. maintaining a same level of treatment delivered to a subject). This may be achieved by using a reduced UV light intensity (using a smaller current) and an increased exposure duration. In this way, smaller amplitude currents can be supplied to the LEDs but over a longer time which can allow more efficient operation whilst still delivering substantially the same amount of UV treatment for a subject.

The above discussion refers to controlling the exposure duration and the UV light intensity to optimise power consumed for treating a subject, responsive to the remaining battery charge being at or below a preset level. In this way, power can potentially be conserved which may extend the battery life. This may allow use of the portable UV sterilising device for providing a greater level of microbial load reduction for the subject (e.g. than otherwise may have been the case) and/or treatment of a greater number of subjects.

In some embodiments of the disclosure, the control unit can be configured to use the at least one ML model to control the exposure duration and the UV light intensity to balance power consumption and total treatment time (i.e. total exposure duration) for treatment of a subject. A total amount of UV treatment that is provided to a subject is dependent on UV light intensity and the duration of the treatment. Use of a higher UV light intensity may allow treatment of a subject to an acceptable microbial load in a shortened time. A shorter total treatment time may be desirable from a user convenience perspective. On the other hand, a shorter total treatment time typically involves use of a higher UV intensity and thus reduced operational efficiency. In the techniques of the present disclosure, exposure duration and UV light intensity may be controlled for a treating a subject so as to achieve a balance between power consumption and total treatment time for that subject.

In some examples, the plurality of selectable modes of operation may comprise a first mode, a second and a third mode each capable of delivering a same amount of UV treatment using different durations and intensities. The first mode may have first treatment duration and first LED UV intensity, the second mode may have a second treatment duration and second LED UV intensity, and the third mode may have a third treatment duration and third LED UV intensity, in which: the first treatment duration is shorter than the second treatment duration, and the second treatment duration is shorter than the third treatment duration; and the first LED UV intensity is greater than the second LED UV intensity, and the second LED UV intensity is greater than the third LED UV intensity. Accordingly, the first mode may be desirable for achieving a shorter treatment time, and the third mode may be desirable for achieving higher operational efficiency, and the second mode may achieve a balance between the first mode and the third mode. In some embodiments of the disclosure, the second mode may be selected for controlling the LEDs according to the second treatment duration and second LED UV intensity which can achieve a balance between power consumption and total treatment time. Moreover, whilst there may be modes that are more optimal in terms of reducing time, or in terms of operational efficiency, a mode that achieves a balance between these aspects may be selected. It will be appreciated that the above discussion provides an example of three modes and that in some examples there may be a plurality of modes between the first and third modes and selection of any of those modes may be possible.

In some examples, the portable UV sterilising device may have a plurality of power-modes. The plurality of power-modes may be selectable by a user so that a user can decide on a power-mode to be used for the portable UV sterilising device. Alternatively or in addition, the plurality of power-modes may be automatically selectable responsive to remaining battery charge. The plurality of power-modes may comprise: i) a power-mode for prioritising optimisation of power consumption; ii) power-mode for prioritising reduction of treatment time; and iii) a power-mode for balancing power consumption and treatment time. Referring again to the above example in which the first, second and third modes are each capable of delivering a same amount of UV treatment using different durations and UV light intensities: when using the power-mode for prioritising optimisation of power consumption, the third mode may be automatically selected for use (i.e. with the smallest LED UV intensity and longest duration); when using the power-mode for prioritising reduction of treatment time, the first mode may be automatically selected for use (i.e. with the largest LED UV intensity and shortest duration); and when using the power-mode for balancing power consumption and treatment time, the second mode may be automatically selected for use.

The above discussion refers to techniques using selectable modes of operation having different UV light intensities and durations. The control unit can be operable to use the at least one ML model to select a mode of operation. The at least one ML model may provide an output which may be indicative of a respective selectable mode for the portable UV sterilising device. For example, classifications may be output by at least one ML model which may correspond to different modes of operation for the portable UV sterilising device. Alternatively, the at least one ML model may be operable to provide an output indicative of an exposure duration and a UV light intensity (e.g. regression values), and the control unit may perform selection of a respective mode based on the ML model output. For example, the control unit may perform a mode selection processing operation (e.g. using software instructions) to select a mode that has a closest match for the ML model output. Alternatively or in addition, the control unit may be operable to control each of exposure duration and UV light intensity according to a continuous scale and the at least one ML model may provide an output (e.g. regression values) for automatically causing the control unit to control the exposure duration and the UV light intensity. Hence more generally, the control unit can be configured to use the at least one ML model for balancing the competing aspects of power consumption and treatment time or prioritising one of reduced power consumption and reduce treatment time.

In some embodiments of the disclosure, at least one ML model may be operable to predict, in dependence on at least one of a type, a quantity, and an initial microbial load for a subject, a required battery charge for treatment of the subject. For example, according to the techniques discussed above a UV light intensity and an exposure duration to be used for treatment of a subject may be used for calculating an amount of required power (e.g. in units Watt-hours, or Amp-hours or similar).

In some embodiments of the disclosure, the control unit may be configured to use at least one ML model to predict whether treatment of the subject is possible for a remaining battery charge for one or more batteries of the portable UV sterilising device. At least one of a type, a quantity and an initial microbial load for a subject may be input to an ML model for predicting required battery charge for treatment of the subject. In addition, remaining battery charge for one or more batteries may also be input to the ML model. The ML model can thus predict whether treatment of the subject is possible for a remaining battery charge. The control unit can be operable to activate the plurality of UV light sources in dependence on whether treatment of the subject is possible for the remaining battery charge. Accordingly, in response to a prediction that treatment will be possible for the subject based on the current battery status, the control unit can activate the plurality of UV light sources. In response to a prediction that treatment will be not possible for the subject based on the current battery status, the control unit may prohibit activation of the plurality of UV light sources. This can contribute to improving food safety. A user that may otherwise have attempted to partially (and thus not successfully) treat a subject may potentially be stopped from attempting partial treatment. In some examples, there may be a manual override function. Furthermore, issues such as insufficient power arising during treatment which may initially go unnoticed by a user can potentially be mitigated. Furthermore, this can encourage treatment of a subject using a single treatment session rather than stopping part way through a treatment session and resuming later which may present issues for food safety. In the case of rechargeable batteries, a user can be prompted to recharge the batteries to a state which will allow successful treatment of the subject using a single treatment session.

In some embodiments of the disclosure, in response to the predicted required battery charge being greater than the remaining battery charge and within a given range of the remaining battery charge, the control unit can be configured to use the at least one ML model to reduce the UV light intensity to allow lower UV light intensity treatment for the subject over an increased exposure duration. In some cases, when the remaining battery charge is smaller than the required battery charge for a treatment, the portable UV sterilising device may reduce the UV light intensity for allowing successful treatment over an extended treatment duration (thereby allowing improved operational efficiency—e.g. due to lower heat associated with the operation of the LED which may also lower power consumption associated with a cooling system). For cases in which the predicted required battery charge and the remaining battery charge are withing a given range (e.g. a few percent of SoC) then the improved operational efficiency associated with reducing the UV light intensity can permit treatment of a subject that otherwise may not have been treated based on the remaining battery charge and use of a higher UV light intensity. In some examples, the ML model may be operable to predict a plurality of different required battery charges for treatment of a subject using different possible UV light intensities and exposure durations. For example, a first (e.g. low), second (e.g. medium) and third (e.g. high) required battery charge may each be predicted (e.g. for the different power-modes). For cases, in which only the first required battery charge is less than the remaining battery charge, then the control unit can control the UV light sources 18 according to a UV light intensity and exposure duration associated with the first required battery charge.

In some embodiments of the disclosure, the control circuitry may use at least one ML model to predict at least one of: change in performance for the portable UV sterilising device; and degradation of one or more components for the portable UV sterilising device. In some examples, one or more first ML models may be used for control of UV light intensity and/or exposure duration based on one or more properties for a subject to be treated, and one or more second ML models may be used for predictive analytics for performance changes and/or and wear of components for the portable UV sterilising device.

Light output of LEDs generally decreases over time such that a same supply current will result in a smaller light output (lower lumen output) as the LED experiences degradation. Light output of LEDs may decrease due to a range of factors including thermal factors and electrical factors. Changes in performance for components such as the UV LEDs themselves and/or the LED drivers for regulating supplied current and voltage may result in changes in light output for the portable UV sterilising device.

Inputs to one or more ML models for predicting change in performance may comprise one or more operating conditions for the portable UV sterilising device. Operation conditions may comprise one or more of: amount of operation (e.g. total hours of use); maximum operating current for LEDs; and average operating current for LEDs. In addition to operating conditions, information such as the total number of UV LEDS, type of the LEDs and/or one or more hardware properties for the LEDs may be used as inputs. Using such inputs, ML techniques can be used to model changes in performance for the portable UV sterilising device. In some examples, digital twin modelling of the portable UV sterilising device may be used. Using AI, a digital twin for the portable UV sterilising device may be simulated. The digital twin may be used to simulate the portable UV sterilising device including specifically simulating the plurality of UV LEDs to predict a total UV light output for the plurality of UV LEDs that accounts for LED degradation over time.

For example, an LED b-rating for the plurality of UV LEDs may be used for the digital twin simulation to factor in a percentage of the LEDs that can be expected to fall below a certain lumen level after a certain operating time.

ML techniques (e.g. digital twin simulation) can be used to predict future changes (decreases) in UV light output by the portable UV sterilising device. In some examples, predicted future decreases in UV light may be used as feedback for controlling the portable UV sterilising device. Specifically, in response to a prediction that a same supply current for an LED will result in a decreased light output at a time in the future, this information may be provided as feedback so that in the future a larger supply current can be used to maintain a same light output to thereby counteract performance degradation. Alternatively, a same supply current may be used but with an extended treatment duration, or both supply current and duration may be controlled to counteract performance degradation and maintain a same treatment effect. Using predicted future changes in performance as feedback for controlling the portable UV sterilising device can ensuring reliable operation and contribute to improving food safety.

In some examples, by forecasting performance decline, in the future one or more control strategies may be implemented which can implement measures to maintain effectiveness of the portable UV sterilising device whilst managing the rate of performance decline. For example, use of lower amplitude currents (and lower UV intensities) over extended treatment durations may be favoured over larger amplitude currents and shorter treatment durations. More generally, by predicting changes in performance, the control unit can be operable to control the portable UV sterilising device to account for changes in performance and extend a useful lifespan of the portable UV sterilising device for treatment of subjects.

In some examples, degradation of one or more components for the portable UV sterilising device may be predicted. This may be used for predicting maintenance for the portable UV sterilising device in advance of component degradation below a set level (e.g. set let of performance or failure). Components such as LEDs, drivers, cooling systems may be modelled (e.g. using digital twin techniques). Degradation of such components can thus be predicted. For example, a time at which performance for one or more components reaches or falls below a set level (e.g. at which the component(s) does not provide satisfactory performance or will fail) can be predicted. Maintenance (e.g. replacement of components) can thus be scheduled in advance of one or more components reaching or falling below the set level. For example, LEDs are generally likely to experience gradual degradation over a period of time rather than a failure. L-values (corresponding to percentages of the initial LED brightness) can be predicted. In some examples, a time at which an L-value will reach a set level (e.g. L90, or L70) can be predicted and used for scheduling maintenance. In some examples, a time at which at least a certain percentage (e.g. 10%, 20%, 30%, 40% or 50%) of the plurality of LEDs will reach a set level of the original light output can be predicted and used for scheduling maintenance.

When used in this specification and claims, the terms “comprises” and “comprising” and variations thereof mean that the specified features, steps, or integers are included. The terms are not to be interpreted to exclude the presence of other features, steps, or components.

The invention may also broadly consist in the parts, elements, steps, examples, and/or features referred to or indicated in the specification individually or collectively in any and all combinations of two or more said parts, elements, steps, examples, and/or features. In particular, one or more features in any of the embodiments described herein may be combined with one or more features from any other embodiment(s) described herein.

It will be appreciated that example embodiments can be implemented by computer software operating on a general purpose computing system. In these examples, computer software, which when executed by a computer, causes the computer to carry out any of the methods discussed above is considered as an embodiment of the present disclosure. Similarly, embodiments of the disclosure are provided by a non-transitory, machine-readable storage medium which stores such computer software.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or encompass equivalents.

Examples or embodiments of the subject matter and the functional operations described herein can be implemented in digital electronic circuitry, or in computer software, firmware, or hardware, including the structures disclosed in this specification and their structural equivalents, or in combinations of one or more of them.

Some examples or embodiments are implemented using one or more modules of computer program instructions encoded on a computer-readable medium for execution by, or to control the operation of, a data processing apparatus. The computer-readable medium can be a manufactured product, such as hard drive in a computer system or an embedded system. The computer-readable medium can be acquired separately and later encoded with the one or more modules of computer program instructions, such as by delivery of the one or more modules of computer program instructions over a wired or wireless network. The computer-readable medium can be a machine-readable storage device, a machine-readable storage substrate, a memory device, or a combination of one or more of them.

The terms “computing device” and “data processing apparatus” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment, or a combination of one or more of them. In addition, the apparatus can employ various different computing model infrastructures, such as web services, distributed computing and grid computing infrastructures.

The processes and logic flows described in this specification can be performed by one or more programmable processors executing one or more computer programs to perform functions by operating on input data and generating output.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices.

1. A portable UV sterilising device comprising:
a proximal portion configured to be held by a user, and a distal portion adjacent the proximal portion;
a plurality of UV light sources positioned at the distal portion and spaced around the distal portion, the plurality of UV light sources configured to project UV light outside the distal portion, wherein when the plurality of UV light sources is activated the plurality of UV light sources is configured to project the UV light radially around substantially all of the distal portion; and
a control unit configured to control at least one of exposure duration and UV light intensity for the plurality of UV light sources, wherein the control unit is configured to use at least one machine learning (ML) model to control at least one of the exposure duration and the UV light intensity,
wherein the portable UV sterilising device has a plurality of modes of operation, at least some of the plurality of modes of operation having at least a different UV light intensity, and
wherein the control unit is configured to use the at least one ML model to select a mode from the plurality of modes to thereby control the UV light intensity.

2. The portable UV sterilising device according to claim 1, wherein the control unit is configured to use the at least one ML model to control at least one of the exposure duration and the UV light intensity in dependence on input data indicative of at least one of: a type of subject to be treated, a quantity of the subject, and an initial microbial load associated with the subject.

3. The portable UV sterilising device according to claim 2, wherein the subject is one of: food; a beverage; and a physical object comprising one or more surfaces.

4. The portable UV sterilising device according to claim 3, wherein the physical object is one of: a laptop device, a mobile phone device, a keyboard device, and a work surface.

5. The portable UV sterilising device according to claim 2, wherein the portable UV sterilising device comprises one or more sensors operable to detect at least some of the input data and/or an input interface operable to receive user input for specifying at least some of the input data.

6. The portable UV sterilising device according to claim 5, wherein the one or more sensors comprise one or more of: an audio sensor operable to detect speech input; and an image sensor operable to capture one or more images, in which at least one of the speech input and the one or more images is indicative of at least one of a type and a quantity for the subject.

7. The portable UV sterilising device according to claim 2, wherein the portable UV sterilising device comprises wireless communication circuitry, wherein the wireless communication circuitry is operable to receive:
at least some of the input data from a mobile user device via a wireless communications network; or
one or more control signals for controlling at least one of the exposure duration and the UV light intensity, the one or more control signals being based on ML model outputs in response to the input data.

8. The portable UV sterilising device according to claim 2, wherein the control unit is configured to use the at least one ML model to control the exposure duration and the UV light intensity differently for at least one of:
i) different food types and/or different beverage types; and/or
ii) different quantities of a same food type and/or different quantities of a same beverage type; and/or
iii) different microbial loads.

9. The portable UV sterilising device according to claim 2, wherein the at least one ML model is operable to predict, in dependence on at least the type and the quantity of the subject, a required battery charge for treatment of the subject.

10. The portable UV sterilising device according to claim 1, wherein the control unit is configured to use the at least one ML model to control at least one of the exposure duration and the UV light intensity in dependence on input data indicative of a remaining battery charge for one or more batteries for powering the portable UV sterilising device.

11. The portable UV sterilising device according to claim 10, wherein in response to the remaining battery charge being at or below a preset level, the control unit is configured to use the at least one ML model to control the exposure duration and the UV light intensity to optimise power consumption for treatment of a subject.

12. The portable UV sterilising device according to claim 10, wherein the control unit is configured to use the at least one ML model to control the exposure duration and the UV light intensity to balance power consumption and total treatment time for treatment of a subject.

13. The portable UV sterilising device according to claim 10, wherein the control unit is configured to use the at least one ML model to predict whether treatment of the subject is possible for the remaining battery charge, and wherein the control unit is operable to activate the plurality of UV light sources in dependence on whether treatment of the subject is possible for the remaining battery charge.

14. The portable UV sterilising device according to claim 10, wherein in response to the predicted required battery charge being greater than the remaining battery charge and within a given range of the remaining battery charge, the control unit is operable to reduce the UV light intensity to allow lower UV light intensity treatment for the subject over an increased exposure duration.

15. (canceled)

16. The portable UV sterilising device according to claim 1, wherein the portable UV sterilising device has a plurality of modes of operation, at least some of the plurality of modes of operation having at least a different exposure duration, and wherein the control unit is configured to use the at least one ML model to select a mode from the plurality of modes to thereby control the exposure duration.

17. The portable UV sterilising device according to claim 1, wherein the control circuitry is configured to use the at least one ML model and/or one or more other ML models to predict at least one of: change in performance for the portable UV sterilising device; and degradation of one or more components for the portable UV sterilising device.

18. The portable UV sterilising device according to claim 17, wherein the control circuitry is configured to use the at least one ML model and/or the one or more other ML models to predict maintenance for the portable UV sterilising device in advance of component degradation below a set level.

19. The portable UV sterilising device according to claim 1; further comprising:
a mobile user device, wherein the mobile user device is operable to communicate with the portable UV sterilising device via a wireless communication, and
wherein at least one of the portable UV sterilising device, the mobile user device, and a server accessible by the mobile user device is operable to run the at least one ML model, and wherein the mobile user device comprises one or more sensors and/or an input interface operable to acquire data for input to the ML model.

20. A method for a portable UV sterilising device, the portable UV sterilising device comprising:
a proximal portion configured to be held by a user, and a distal portion adjacent the proximal portion;
a plurality of UV light sources positioned at the distal portion and spaced around the distal portion, the plurality of UV light sources configured to project UV light outside the distal portion, wherein when the plurality of UV light sources is activated, the plurality of UV light sources is configured to project the UV light radially around substantially all of the distal portion; and
a control unit configured to control at least one of exposure duration and UV light intensity for the plurality of UV light sources, wherein the method comprises:
controlling at least one of exposure duration and UV light intensity for the plurality of UV light sources using at least one machine learning (ML) model.

21. A portable UV sterilising device comprising:
a proximal portion configured to be held by a user, and a distal portion adjacent the proximal portion;
a plurality of UV light sources positioned at the distal portion and spaced around the distal portion, the plurality of UV light sources configured to project UV light outside the distal portion, wherein when the plurality of UV light sources is activated the plurality of UV light sources is configured to project the UV light radially around substantially all of the distal portion; and
a control unit configured to control at least one of exposure duration and UV light intensity for the plurality of UV light sources, wherein the control unit is configured to use at least one machine learning (ML) model to control at least one of the exposure duration and the UV light intensity,
wherein the portable UV sterilising device has a plurality of modes of operation, at least some of the plurality of modes of operation having at least a different exposure duration, and
wherein the control unit is configured to use the at least one ML model to select a mode from the plurality of modes to thereby control the exposure duration.
